(19) United States
(12) Patent Application Publication
(10) Pub. No.: US 2020/0107746 A1
(43) Pub. Date: Apr. 9, 2020

(54) Conductive Adhesive Polymers with Modifiable Properties

(71) Applicants: Wayne Cooper Smith, Reno, NV (US); Billie Adams, Reno, NV (US)

(72) Inventors: Wayne Cooper Smith, Reno, NV (US); Billie Adams, Reno, NV (US)

(21) Appl. No.: 16/595,332

(22) Filed: Oct. 7, 2019

Related U.S. Application Data
(60) Provisional application No. 62/742,392, filed on Oct. 7, 2018.

(51) Int. Cl. A61B 5/0408; C09J 9/02; C09J 11/06

(52) U.S. Cl. CPC A61B 5/04087; C09J 9/02; A61B 2562/0209; C09J 2205/31; C09J 11/06

(57) ABSTRACT

A Medical electrode is disclosed. It comprises a conductive adhesive, such as a polymer adhesive, that can switch from a high attachment phase to a low attachment phase when exposed to an initiator. The initiator can be electrical, chemical, mechanical, or photolytic in nature. The ability to switch phases allows the adhesiveness of the electrode to be changed depending upon whether the electrode is being attached to a patient, whether the electrode is operational, or whether the electrode is being detached. Use of such materials can allow less glue residue to be left by the electrodes, which allows for easier cleanup and in some cases much less painful electrode removal.

### CROSS-REFERENCE TO RELATED APPLICATION(S)

The present application is related to and claims priority to U.S. Provisional Patent Application No. 62/423,392, filed Oct. 7, 2018, pending, which is incorporated by reference in its entirety.

### BACKGROUND OF THE INVENTION

Electrically conductive pastes are used for many applications in the medial field. Often, the conductive paste must be used with a second paste or glue that acts as an adhesive. Many medical testing devices or instruments depend on the use of an electrically conductive material to carry electrical signal from the patient's body to one or more electrodes attached to the device or instrument. A few of these medical testing procedures are electroencephalogram (EEG), electrocardiogram (EKG), and electromyography (EMG). To reduce patient recovery time after muscle strain or surgery, physical therapy often uses electrical muscle stimulation (EMS) or neuromuscular electrical stimulation (NMES). Electrically conductive pastes are also used in physical therapy applications. The electrical signal is carried from the device or instrument through electrodes and through a conductive material to the patient's body.

All these applications require the use of highly electrically conductive material with an adhesive material. Currently, all conductive materials or pastes have low adhesive properties, or the adhesive properties of the pastes is significantly reduced due to an unwanted interaction with moisture, which negatively impacts the adhesion to the patient's body. To overcome the low adhesion limitation, a paste or glue with high adhesion properties is used with the conductive material. One significant drawback with using the pastes or glues with high adhesion is the later removal of the conductive and the adhesive material. Today, harsh solvents are used to dissolve the glue or pastes, which often irritates the patient's skin. Application site cleanup is also time-consuming for medical technicians.

Currently for EEG testing, small electrodes are attached to the patient's scalp using a multiple step process. After the EEG testing is complete, the electrodes are forcefully removed from the patient's scalp and the conductive adhesive is dissolved with acetone. Both the process of attaching and detaching the electrodes and the process of removal of the residual adhesive from the patient's scalp is painful and time-consuming.

Also, for EKG testing, the electrodes are attached to a metal conductive node on the top side of a small patch. The other side of the patch has a conductive adhesive that contacts the patient's body around the chest area to monitor the electrical signals of the heart. Again, both the process of attaching and detaching the electrodes and the process of removal of the residual adhesive from the patient's scalp is painful and time-consuming.

When a patient is required to get an EEG, twenty-eight electrodes are attached to the patient scalp. The first step in the process is to rough up the area of the patient's scalp where the electrode is to be attached. The roughing process consists of the medical technician using sandpaper to abrasively remove any unwanted dead skin and foreign material to promote the adhesion of the electrodes to the scalp. In the second step of the process of attaching the electrodes, the medical technician places a large amount of glue on each electrode. Each electrode is attached to the scalp of the patient. After each of the twenty-eight electrodes are attached to patient's scalp, the medical technician injects a conductive gel at each of the twenty-eight sites where the electrodes are attached to the scalp. Due to the low adhesive properties of the glue, a mess cap must be placed on the patient's scalp to hold the electrodes in intimate contact with the scalp. In the process of removing the electrodes, the medical technician forcefully removes all the electrodes and acetone is applied to the scalp with a piece of cotton or medical wipe to dissolve the glue. The time required to forcefully remove the electrodes is short, but after the electrodes are removed, the time required for the medical technician to remove the glue from each of the sites is significant because the acetone takes approximately an additional one to two minutes per location to dissolve the glue.

The process of attaching the electrodes takes approximately one hour and the process of detaching the electrodes takes approximately one hour, which is not comfortable for any patient. The Acetone used to dissolve the glue often results in significant pain and irritation to the patient because the scalp is already tender and irritated by the initial roughing process.

When a patient is required to get EKG testing, the electrodes are attached to a metal conductive node on the top side of a small patch. The other side of the patch has a conductive adhesive that contacts the patient's body around the chest area to monitor the electrical signals of the heart. During the medical procedure, the most common complication is that the electrodes do not stay in contact with the patient's body. The most common cause of the electrode adhesion failure is the nervous patient's perspiration. The perspiration or any kind of moisture negatively impacts the adhesion properties of the glue. Improper adhesion of the electrodes reduces the conductivity or attenuates the electrical signal resulting in low quality of the test. For the electrodes to be detached, the medical technician has to forcefully pull the probes from the patient's body. Removing the electrodes after the EKG test is not time-consuming. But it is painful.

### SUMMARY

In view of the foregoing disadvantages inherent in the known medical art, the present disclosure provides a novel medical electrode. The general purpose of the present disclosure, which will be described subsequently in greater detail, is to provide a medical electrode.

A medical electrode is disclosed. The medical electrode comprises a patch, a lead connector disposed on the patch or in the patch, and a skin adhesive disposed on the patch. The skin adhesive is in electrical connection with the lead connector and has a high attachment phase and a low attachment phase and can respond to an initiator that causes the adhesive to switch between the high attachment phase and low attachment phase. In some embodiments the initiator is a chemical, electrical, thermal, or photolytic initiator. In some of these embodiments, the thermal initiator supplies a change in temperature—either an increase or decrease. In some embodiments, the thermal initiator causes a rapid decrease in temperature and is a material such as chloroethane. UV light is a useful photolytic initiator.

In these or other embodiments the high attachment phase has a stickiness or internal strength higher than the stickiness or internal strength of the low attachment phase.

In some embodiments the adhesive comprises a polymer such as a conductive polymer or the adhesive comprises a polymer mixed with a conductive material. Various methods of using or attaching the medical electrode are also disclosed

For purposes of summarizing the invention, certain aspects, advantages, and novel features of the invention have been described. It is to be understood that not necessarily all such advantages may be achieved in accordance with any one particular embodiment of the invention. Thus, the invention may be embodied or carried out in a manner that achieves or optimizes one advantage or group of advantages as taught without necessarily achieving other advantages as may be taught or suggested. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a surface staining composition and method, constructed and operative according to the teachings of the present disclosure.

FIG. 1 depicts a medical electrode according to an embodiment of the invention.

FIG. 2 depicts a method of using a medical electrode according to an embodiment of the invention.

FIG. 3 depicts a method of using a medical electrode according to an alternative embodiment of the invention.

FIG. 4 depicts a method of using a medical electrode according to an alternative embodiment of the invention.

FIG. 5 depicts a method of using a medical electrode according to an alternative embodiment of the invention.

The various embodiments of the present invention will be described with the appended drawings, wherein like designations denote like elements.

### DETAILED DESCRIPTION

The terminology used is for the purpose of describing example embodiments only and is not limiting. As used, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described do not necessarily requiring their performance in the order discussed or illustrated, unless so stated. Additional or alternative steps may be employed,

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on”, “directly engaged to”, “directly connected to” or “directly coupled to” another element or layer, there may be no intervening elements or layers present. Other words used to describe the relationship between elements should be interpreted in a like fashion (e.g., “between” versus “directly between”, “adjacent” versus “directly adjacent”, etc.). As used, the term “and/or” includes any and all combinations of one or more of the associated listed items.

Although this document uses the terms first, second, third, etc. to describe various elements, components, regions, layers, and sections, these terms don't limit those elements, components, regions, layers, and sections. This usage is to distinguish one element, component, region, layer or section from another region, layer, or section. Terms such as “first”, “second”, and other numerical terms when used do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath”, “below”, “lower”, “above”, “upper” and the like, may be used for ease of description to describe one element or feature's relationship to another element(s) or feature(s) as illustrated in the figures. Spatially relative terms may encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used interpreted accordingly.

This invention is an electrically conductive adhesive where the adhesive properties can be modified as needed. During the attachment of the electrodes to the body, the adhesive force is high enough to hold the electrodes in intimate skin contact. During the detachment process the adhesive force will be reduced to facilitate the removal of electrodes. This reduces the discomfort of the patient and reduces the attenuation of the electrical signals, resulting in higher quality electrical signal while improving the ability of the medical technicians to perform the testing in a timely manner.

According to various embodiments of the invention, a conductive adhesive is used to attach medical electrodes to skin to conduct a medical procedure requiring measuring physiological potentials. The adhesives demonstrate a high attachment phase, which is defined as a phase in which the various adhesive parameters useful for securing the electrodes to skin combine to create an overall adhesive force, stickiness, etc. that is high during installation of the electrodes in some embodiment. The adhesives also demonstrate a low attachment phase, which is defined as a phase in which the various adhesive parameters useful for securing the electrodes to skin combine to create an overall adhesive force, stickiness, etc. that is low during removal of the electrodes after their use. An external stimulus or initiator causes the adhesives to switch from a high attachment phase to a low attachment phase in some embodiments. In other embodiments, an external stimulus or initiator causes the adhesives to switch from a low attachment phase to a high attachment phase.

That is, after the testing or medical treatment process completes and during electrode detachment, the adhesive force decreases significantly.

In some embodiments, the natural state of the conductive material will be liquid or paste with relatively low dynamic viscosity (below 2.0×10² Pa×s). In some embodiments the combined mechanical and chemical adhesive force will be relatively high (greater than 10 grams/mm) and the resistance will be less than 5 mohms/mm². Other parameters, such as internal strength contribute to how well the adhesive will function to adhere the electrode to skin.

During the detachment process, the combined mechanical and chemical adhesive force contributing to the high attachment phase will be reduced through exposure to a stimulus or initiator such as a chemical initiator, mechanical initiator, photolytic initiator, thermal initiator, etc. More specifically, the stimulus or initiator can result in heating or cooling of the adhesive to change it from a high attachment phase to a low attachment phase. Alternatively, photolytic initiation is initiation such as exposing the adhesive to appropriate light or other electromagnetic radiation such as UV light, or by exposure to some other radiation treatment or a combination of these. In some embodiments, thermal initiation can occur by exposing the adhesive to infrared light, which will cause the material's temperature to increase. One goal is for the adhesive to have high adhesive strength while in the high attachment phase without interfering with the necessary electrode properties of the material. As an example, in some embodiments the adhesive on the electrode is warmed to make it stickier and then the electrode is placed on the skin. In these or other embodiments, as the adhesive cools, it grips more strongly to the skin and the medical procedure is conducted. Afterwards, rewarming the adhesive allows it to be more easily removed. In another example, the adhesive has strong adhesive properties yielding a high attachment phase when the adhesive has a temperature near the temperature where the clinician will conduct the medical treatment. After treatment, the adhesive is treated with a substance that cools it rapidly causing it to lose its adhesive quality and therefore to easily detach from the skin. One such substance is chloroethane.

Another embodiment of this invention is to apply electrodes when the conductive adhesive is in a low attachment phase, where the adhesive has a relatively low adhesive force, and after attachment applying an initiator to cause the adhesive to switch to a high attachment phase. Said another way, after the electrodes have been attached, the electrodes are treated or subjected to a stimulus or initiator that causes the combined mechanical and chemical adhesive force to increase temporarily or until the adhesive is treated or subjected to an additional stimulus or initiator

Conductive adhesives can be intrinsically conductive. This means that the material of the adhesive is also conductive. For instance, if the adhesive were a polymer, the polymer itself would be conductive. Alternatively, adhesives can be modified to be conductive by adding conductive material, such as by mixing the conductive material into the adhesive.

In some embodiments the conductive material uses conductive particles such as aluminum, silver, iron, copper, carbon, such as graphite or conductive carbon fiber particles, noble metals, or other conductive particles. Especially useful are conductive materials that are not known to cause reactions when topically exposed to skin.

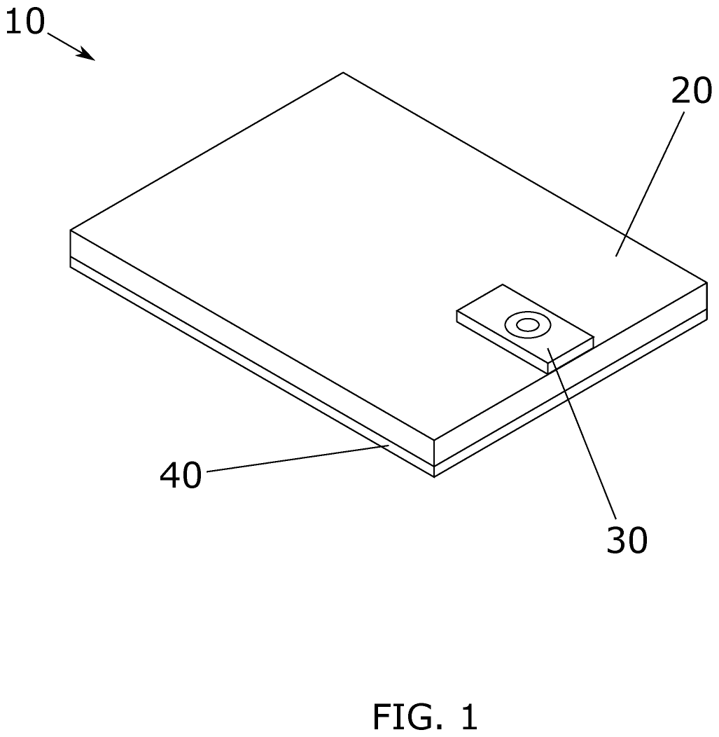

FIG. 1 depicts medical electrode 10, which comprises patch 20, lead connector 30, and a layer 40 of skin adhesive. The exact structure and geometry of medical electrode 10 depends upon the specific medical test or treatment that electrode 10 is to be used with. Also, the nature of the medical test or experiment affects the specific characteristics of patch 20, as well. The overall function of medical electrode 10 is to adhere to skin and provide an electrical connection between the medical test or treatment instrument and the skin. The main function of patch 20 is to provide a substrate to mount lead connector 30, which is the direct connection between electrode 10 and the instrument. Layer 40 adheres the patch to the skin and also provides a conductive path between the skin and the remainder of electrode 10.

Two primary features of medical electrode 10 are good adherence to the skin and providing good, substantially constant, electrical connectivity or conductivity between the skin and the instrument. In most cases, “good” conductivity means that the electrical potential of the skin at the position of medical electrode 10 is accurately transmitted through electrode 10 with little enough resistance that the instrument can accurately measure the potential of the skin or at least measure a potential proportional to the potential of the skin at the position of electrode 10. Constant electrical connectivity, for purposes of this disclosure, means that the electrical connection is not hampered by intermittent electrical disconnection.

Patch 20 can comprise varied materials as is well known to those of ordinary skill in the art. For instance, patch 20 can be a woven material, such as a traditional bandage, or can be paper, plastic, etc.

Lead connector 30 can be any number of well-known lead connectors. In some cases, this can be an electrical wire running into the patch or adhesive layer glued in place or can be one side of a metallic snap, or any other connection for patches or electrodes that are well known in the art.

The adhesive functions to adhere patch 20 to the skin during the experiment. Therefore, layer 40 should have enough adhesive strength during the medical treatment or test to retain patch 20 against the skin in good electrical contact. Also, skin adhesive layer 40 should be easy to remove after the treatment or test.

Inventive adhesives exhibit at least two phases or conditions. For purposes of this disclosure, referring to an adhesive as having a “phase” means that at some point the adhesive is exhibiting various adhesive properties that make it function in the way described. For instance, an adhesive that is in “a high attachment phase” is an adhesive with an assortment of properties that cause the adhesive to provide good adherence to the skin. The parameters that support or contribute to high adhesive strength or into causing a high attachment phase are well-known in the art.

Similarly, a low attachment phase for an adhesive means that the adhesive has an assortment of parameters causing the adhesive to be less sticky or to adhere to the skin less strongly than in the high attachment phase. While having high adhesive strength such as in the high attachment phase is useful for mounting electrode 10 during the procedure, having lower adhesive strength or being in a low attachment phase is useful after the medical test or procedure when the electrodes are being removed from the skin.

Invention adhesives include adhesives that can transition between a high attachment phase and a low attachment phase when exposed to a stimulus or an initiator. For purposes of this disclosure, an “initiator” is generically any stimulus that causes an invention adhesive to switch between these phases. For instance, certain adhesives are known to cure when exposed to UV light. Therefore, before exposure these adhesives are liquids or gels with low adhesive strength or that are in the low attachment phase. As a practical matter, in some embodiments, adhesives in the low attachment phase are adhesives that may have little enough adhesive strength that they do not adhere patch 20 to skin at all or they are adhesives that have an adhesive strength lower than the adhesive strength desired for electrode 10 during the procedure. Returning to the UV activated example discussed above, the stimulus is exposure to UV light. That is, UV light is a photolytic initiator because it involves application of the stimulus, UV light, which causes the adhesive properties to change. In this case, exposure to UV light cures the adhesive by cross-linking it, transforming it from a minimally adhesive gel to a strongly adherent cross-linked form.

Various embodiments of the invention contemplate initiators that are chemical, thermal, mechanical, photolytic, or electrical, or that expose the adhesive to various radiations or other stimuli that cause a switch between a low attachment phase and a high attachment phase. Those of ordinary skill in the art recognize many adhesives that can change their adhesive strength, that is, switch between a low or high attachment phase and the opposite attachment phase, upon receiving a myriad of different stimuli.

The ability to transition between a low attachment phase and a high attachment phase or a high attachment phase and the low attachment phase is important for the function of the adhesive. But the adhesive must also be conductive. For purposes of this invention, the adhesive can be inherently conductive, which means that the adhesive material itself conducts electricity. That is, the material that causes adhesion is the same material that conducts electricity. In other embodiments, an adjuvant is added to the adhesive to make the adhesive conductive. For instance noble metal powders, such as silver, gold, or platinum when added to the adhesive in appropriate ratios allow the adhesive to conduct electricity because the electrons move from metal particle to metal particle—because the metal particles contact each other and provide a complete conductive path through the adhesive. Thus, the concentration or distribution of metal particles in the adhesive determines the conductivity of the adhesive. In some cases, the more metal particles available or included in the adhesive, the higher the conductivity. But also, in some cases, the more non-adhesive material mixed into the adhesive, the lower the adhesive properties. So those of ordinary skill in the art recognize that making an adhesive conduct electricity in this way is a balancing act between enough conductivity and enough adhesive strength. The necessity of doing this balancing act means that, in some embodiments, an adhesive that is intrinsically conductive would perform better in some ways. But intrinsically conductive adhesives are much less common than metal particles.

Many modern adhesives are polymeric in nature. Of course, this includes polymers of various chain length and branching and includes rubbers. Some of these polymeric adhesives are intrinsically conductive. Despite that, intrinsically conductive polymers are not common, but a great deal of research is continuously being focused on such materials. In some embodiments, there is no fundamental reason why any future intrinsically conductive polymer would not acceptably function as skin adhesive 40 in embodiments of the current invention.

Specific classes of adhesives or polymeric adhesives useful in various embodiments of the current invention include adhesives with a silicone base that are high viscosity liquid polymers. Some of these materials have high tackiness. These materials can be modified with conductive powders or fibers or other material such as noble metal powders, graphite, highly conductive carbon fibers, and other metal powders. In some embodiments the metal powder is selected such that the material is not known to cause topical skin reactions or induce allergic reactions when applied to skin. In other embodiments, the metal powder is chosen so that the overall adhesive combination, metal powder mixed with adhesive, is stable enough to provide a reasonable or economically feasible shelf life for the electrodes. Other specific examples include pressure sensitive adhesive tapes in which the adhesive is UV curable. Placed in the framework described in this disclosure, those tapes would transition from a low attachment phase to a high attachment phase upon exposure to a photolytic initiator, in this case UV light. Another useful class of conductive adhesives are those adhesives that have a high tack, but when exposed to a higher temperature, bonds in the mixture breakdown or loosen making the adhesive have less adhesive force after the material is warmed. Described in accordance with the current disclosure this class of materials would transition from a high attachment phase to a low attachment phase upon exposure to an initiator, a thermal initiator that supplies increased heat.

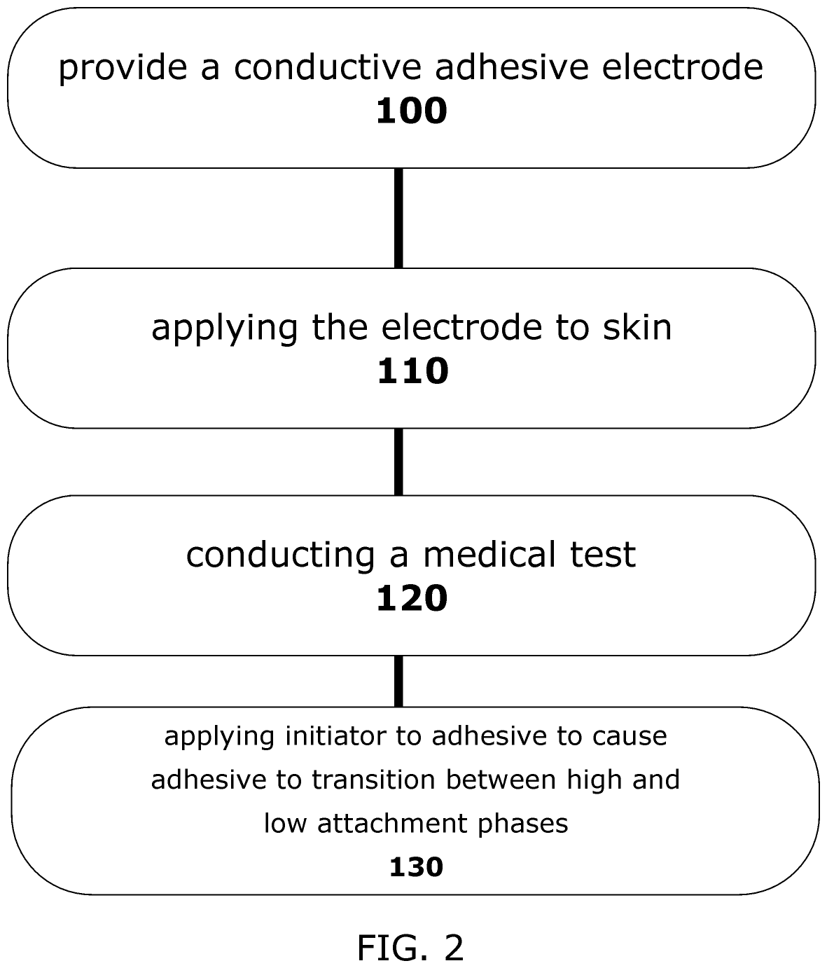

FIG. 2 depicts a method of using medical electrode 10 of an embodiment of the current invention. Step 100 is to provide a conductive adhesive electrode. This electrode is applied to the skin in step 110. In some embodiments this is before conducting the applicable medical test. Step 120, at some point after the test, when it is time to remove medical electrode 10, an initiator is applied to electrode 10, patch 20, or directly to adhesive 140, which causes the adhesive to transition between a low attachment phase and a high attachment phase. In some cases, in the high attachment phase, the adhesive layer is rubbery and sticky, but when the initiator or stimulus is applied to the adhesive or electrode 10, a transition to a low attachment phase occurs because the adhesive material becomes brittle or less sticky causing it to cease adhering to skin.

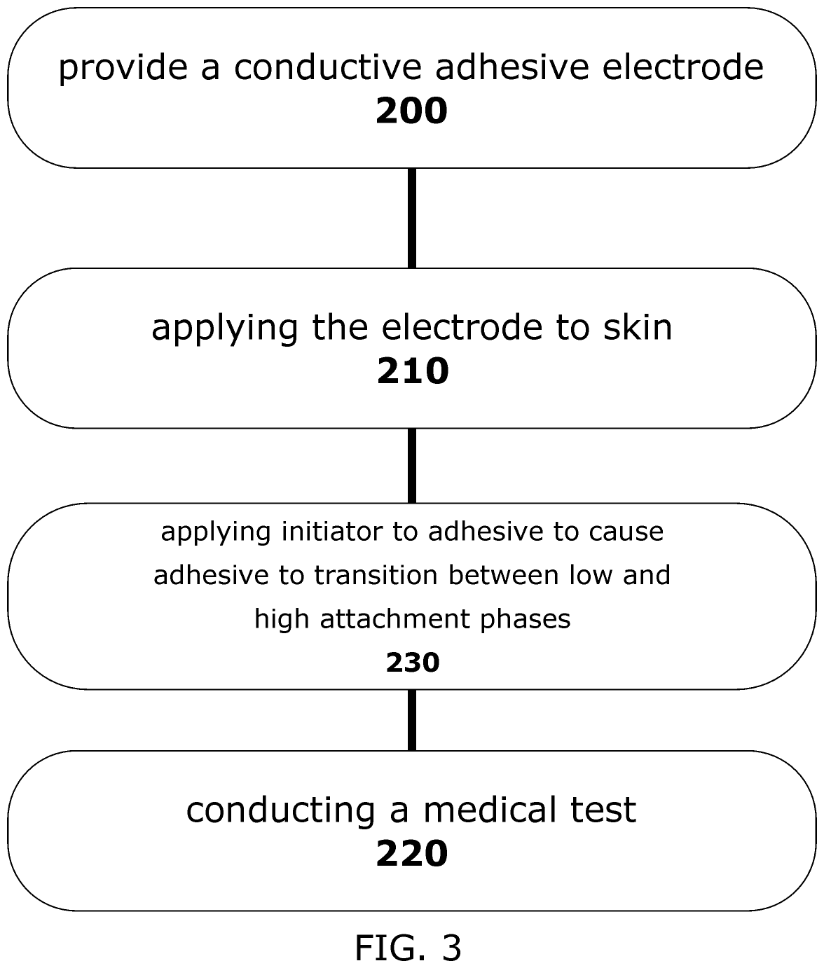

FIG. 3 depicts a method of using medical electrode 10 is an embodiment of the current invention. Step 200 in this method is to provide a conductive adhesive electrode, as before. Then, the electrode is applied to the skin, step 210. In this embodiment, the initiator is applied to the adhesive to cause the adhesive to transition between a low attachment phase and a high attachment phase. This ensures that the electrode 10 remains secured during the medical test. After electrode 10 is securely adhered to the skin, the step of conducting a medical test, step 220, is completed.

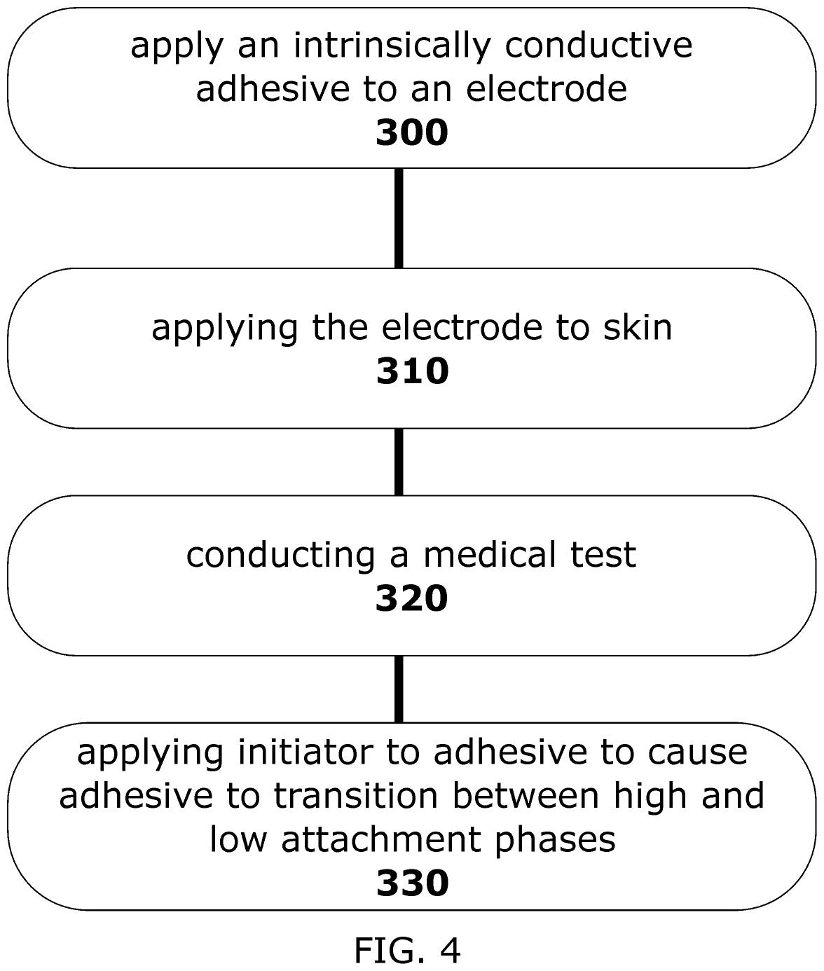

FIG. 4 depicts a method of making or using medical electrode 10 according to yet another embodiment of the current invention. Step 300 comprises applying an intrinsically conductive adhesive to an electrode, typically patch 20 of electrode 10. After that, the electrode is applied to skin in step 310, and a medical test or procedure is conducted in step 320. After the medical test, electrode 10 or adhesive layer 40 is exposed to an initiator that causes the adhesive mixture to transition between the high attachment phase and the low attachment phase, step 330.

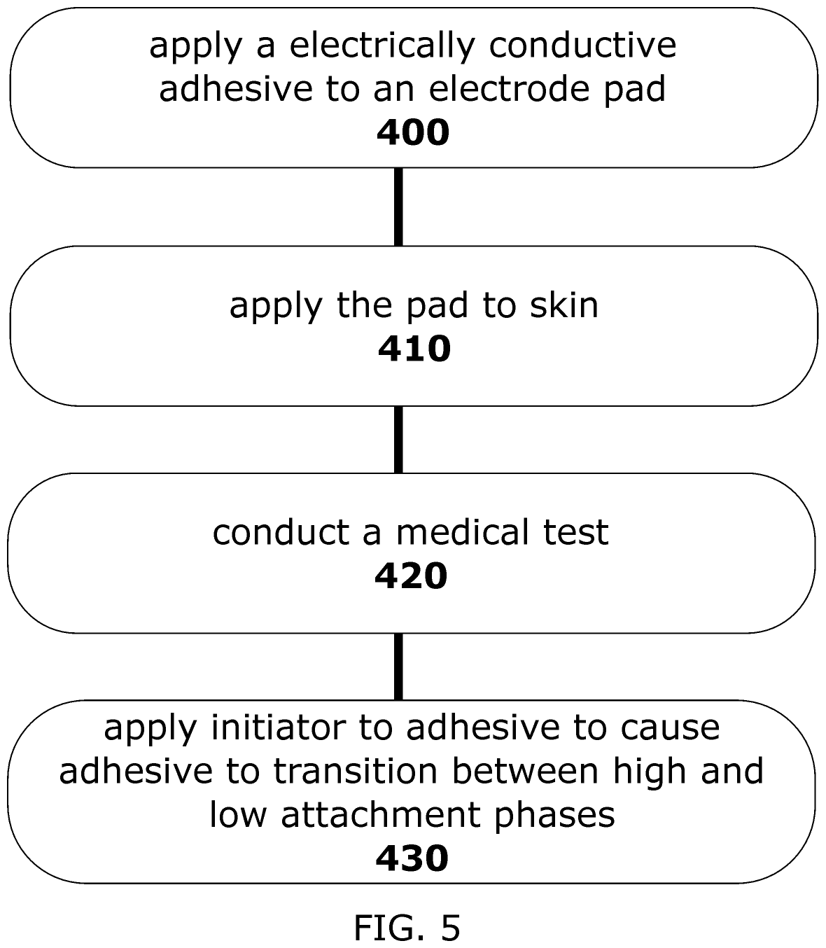

FIG. 5 depicts a method of making or using medical electrode 10 according to an embodiment of this invention, as well. Step 400 comprises applying an electrically conductive adhesive to an electrode patch. The adhesive can be intrinsically conductive or can be a mixture of components at least one of which causes electrical conductivity in the mixture. Next, in step 410, patch 20 is applied to the skin. In step 420, a medical test is conducted and when the time to remove the medical electrode from the skin comes, in step 430, an initiator is applied to the adhesive mixture to cause the mixture to transition between the high attachment phase and the low attachment phase.

Those of ordinary skill in the art will recognize that there are other methods of making and using invention medical electrodes 10.

This invention contemplates embodiments with adhesive mixtures that contain components that make the adhesive act as an adhesive, components that cause the adhesive to be conductive, components that cause the mixture to transition between a low attachment phase and a high attachment phase upon application of a stimulus or initiator, and components that cause the adhesive to transition between a high attachment phase and a low attachment phase upon application of a stimulus or initiator. Alternatively, this invention contemplates embodiments with adhesive mixtures that contain components that make the adhesive act as an adhesive, components that cause the adhesive to be conductive, components that cause the mixture to transition between a low attachment phase and a high attachment phase upon application of a stimulus or initiator, or components that cause the adhesive to transition between a high attachment phase and a low attachment phase upon application of a stimulus or initiator. Yet other embodiments have adhesive mixtures that contain components that make the conductive adhesive act as an adhesive, components that cause the mixture to transition between a low attachment phase and a high attachment phase upon application of a stimulus or initiator, and components that cause the adhesive to transition between a high attachment phase and a low attachment phase upon application of a stimulus or initiator. Yet other embodiments have adhesive mixtures that contain components that make the conductive adhesive act as an adhesive, components that cause the mixture to transition between a low attachment phase and a high attachment phase upon application of a stimulus or initiator, or components that cause the adhesive to transition between a high attachment phase and a low attachment phase upon application of a stimulus or initiator.

The stimuli or initiator in each case can be the same or different. The component that causes the transition from the low attachment phase to the high attachment phase can use one set of materials that respond to one type of initiator, and the component that causes the transition from high attachment phase to low attachment phase can use a set of the same or different components that respond to the same or different type of initiator. Or the component that cause the transitions can use similar types of initiators.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

The embodiments of the invention described are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

It should be noted that the steps described in the method of use can be carried out in many different orders according to user preference. The use of “step of” should not be interpreted as “step for”, in the claims and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for making medical electrodes [NOTE: e.g., different step orders within above-mentioned list, elimination or addition of certain steps, including or excluding certain maintenance steps, etc.], are taught.

What is claimed is new and desired to be protected by Letters Patent is set forth in the appended claims:

1. A medical electrode comprising:
a patch;
a lead connector disposed on the patch; and
a skin adhesive disposed on the patch in electrical connection with the lead connector, wherein the skin adhesive has a high attachment phase and a low attachment phase, and wherein an initiator causes the adhesive to switch between the high attachment phase and the low attachment phase

2. The electrode of claim 1 wherein the initiator is a chemical, thermal, or photolytic initiator.

3. The electrode of claim 2 wherein the thermal initiator supplies decreased temperature or increased temperature.

4. The electrode of claim 3 wherein the thermal initiator is chloroethane.

5. The electrode of claim 4 wherein the high attachment phase has a stickiness or internal strength higher than the stickiness or internal strength of the low attachment phase.

6. The electrode of claim 5 wherein the adhesive comprises a polymer.

7. The electrode of claim 6 wherein the polymer is conductive.

8. The electrode of claim 7 wherein the polymer comprises conductive material.

9. The electrode of claim 2 wherein the photolytic initiator is UV light.

10. The electrode of claim 9 wherein the polymer is conductive.

11. A method of attaching medical electrodes comprising:
providing a medical electrode comprising:
a patch;
a lead connector disposed on the patch; and
a skin adhesive disposed on the patch in electrical connection with the lead connector, wherein the skin adhesive has a high attachment phase and a low attachment phase, and wherein an initiator causes the adhesive to switch between the high attachment phase and the low attachment phase

12. The method of claim 11 further comprising applying the patch to a patient's skin.

13. The method of claim 12 further comprising conducting a medical test.

14. The method of claim 13 followed by applying an initiator to the adhesive to cause the adhesive to transition between the high attachment phase and the low attachment phase.

15. The method of claim 14 wherein the initiator is a chemical, thermal, or photolytic initiator.

16. The method of claim 15 wherein the thermal initiator supplies decreased temperature or increased temperature.

17. The method of claim 16 wherein the thermal initiator is chloroethane.

18. The method of claim 17 wherein the high attachment phase has a stickiness or internal strength higher than the stickiness or internal strength of the low attachment phase, the adhesive comprises a polymer, and the polymer is conductive.

19. The method of claim 13 further comprising applying an initiator to the adhesive to cause the adhesive to transition between the low attachment phase and the high attachment phase.

20. The method of claim 19 wherein the adhesive comprises a conductive polymer.
